(19) United States
(12) Patent Application Publication
(10) Pub. No.: US 2014/0276407 A1
(43) Pub. Date: Sep. 18, 2014

(54) Medical Devices Having Micropatterns

(71) Applicant: BOSTON SCIENTIFIC SCIMED, INC., Maple Grove, MN (US)

(72) Inventors: Robert B. DeVries, Northborough, MA (US); Samuel Raybin, Marlborough, MA (US); James Weldon, Newton, MN (US); Claude O. Clerc, Marlborough, MA (US); Gary J. Leanna, Holden, MA (US); Gary S. Kappel, Acton, MA (US); Meghan Elizabeth Soens, Lexington, MA (US); Paul Smith, Smithfield, RI (US)

(73) Assignee: BOSTON SCIENTIFIC SCIMED, INC., Maple Grove, MN (US)

(21) Appl. No.: 14/210,896

(22) Filed: Mar. 14, 2014

Related U.S. Application Data
(60) Provisional application No. 61/798,545, filed on Mar. 15, 2013.

(51) Int. Cl. A61M 25/10; A61B 17/22

(52) U.S. Cl. CPC A61M 25/10; A61B 17/22032
USPC 604/103.08; 606/127

(57) ABSTRACT

Medical devices having one or more micropatterned surfaces (e.g., micropatterned coatings).

### CROSS REFERENCE TO RELATED APPLICATIONS

This application claims the benefit of provisional U.S. Patent Application Ser. No. 61/798,545 (entitled MEDICAL DEVICES HAVING MICROPATTERNS, filed on Mar. 15, 2013), which is hereby incorporated by reference in its entirety.

The following commonly assigned patent applications are incorporated herein by reference, each in its entirety:
U.S. Pat. App. Ser. No. 61/798,685 (Firstenberg et al.), entitled ANTI-MIGRATION MICROPATTERNED STENT COATING, filed on Mar. 15, 2013 (Atty. Docket No. 563.2-15576U502);
U.S. Pat. App. Ser. No. 61/798,897 (Seddon et al.), entitled ANTI-MIGRATORY STENT COATING, filed on Mar. 15, 2013 (Atty. Docket No. S63.2-15705US01);
U.S. Pat. App. Ser. No. 61/798,794 (Clerc), entitled DELIVERY DEVICE FOR PARTIALLY UNCONSTRAINED ENDOPROSTHESIS, filed on Mar. 15, 2013 (Atty. Docket No. 563.2-15804US01);
U.S. Pat. App. Ser. No. 61/799,312 (Fleury et al.), entitled SUPERHYDROPHOBIC COATING FOR AIRWAY MUCUS PLUGGING PREVENTION, filed on Mar. 15, 2013 (Atty. Docket No. 563.2-15857US01); and
U.S. Pat. App. Ser. No. 61/798,991 (Bertolino et al.), entitled BIOPSY TOOL HAVING MICROPATTERN, filed on Mar. 15, 2013 (Atty. Docket No. S63.2-15935US01).

### BACKGROUND

A wide variety of medical devices are known in the art. Some medical devices include a part that slidingly contacts another part of the medical device and/or biological tissue. As a consequence of material selection, some sliding contacts between materials experience unduly high friction, resulting in the need for greater forces to cause the sliding contacts. However, the friction to be overcome is not always a constant throughout the range of motion of a particular device part, resulting in difficulty in operation of the device due to imprecise and unpredictable changes in friction to be overcome. Since variations in friction may not be expected, a user of the medical device may inadvertently apply too much force to a device part, resulting in the movement of the device part outside of the desired range of motion. Movement of some device parts (e.g., needles, etc.) outside of the intended range of motion may result in reduced efficiency or patient harm.

Fine needle aspiration (FNA) needles are used in, for example, diagnostic procedures to investigate superficial (just under the skin) and deeper lumps or masses. The FNA needles are thin, hollow needles inserted into a mass for obtaining samples of cells for later cytology and/or histological examination. Both the inside and outside of an FNA needle including a sliding contact with tissue during, for example, a biopsy.

Some research in the area of micropatterns has been conducted by, for example, Hoowaki, LLC (www.hoowaki.com) (e.g., designs and creates microsurfaces to control surface friction and surface tension, micropatterned extrusion, curved micropatterned tooling, metal and polymer surfaces, etc.) and nanoGriptech, Inc. (www.nanogriptech.com) (gecko-inspired dry microfibrillar adhesives, slippage control, micropatterns on pills, etc.).

As described above, properly dimensioned patterned surfaces may render a material superhydrophobic and/or fluid repellent. In many applications (e.g., medical device applications) the contact of a surface with liquids has been problematic due to fouling and/or accumulation of biological material that may increase the likelihood of infection or may cause other biological responses. Medical devices having surfaces with improved properties (e.g., wetting properties) are desired.

Additional details regarding wetting and superhydrophobes may be obtained from “Wetting,” Wikimedia Foundation, Inc., last modified Feb. 23, 2013 (available online at http://en.wikipedia.org/wiki/Wetting, last accessed Mar. 8, 2013) and “Superhydrophobe,” Wikimedia Foundation, Inc., last modified Feb. 20, 2013 (available online at http://en.wikipedia.org/wiki/Superhydrophobe, last accessed Mar. 8, 2013) (describing potential applications in textiles and micro fuel cell chips), each of which is incorporated by reference in its entirety. Research regarding interaction of surfaces with liquids has been reported. (See, e.g., Chang et al., “Design and fabrication of a nanostructured surface combining antireflective and enhanced hydrophobic effects,” Nanotechnology, 18 Jul. 2007, 18(28):285303; Bico et al., “Pearl Drops,” EPL (Europhysics Letters), 1999, 47(2):220-226; Bravo et al., “Transparent superhydrophobic films based on silica nanoparticles,” Langmuir, 2007 Jun. 19, 23(13):7293-7298; Sayer et al., “The Influence of Hydrophobic Windshield Coating on Driver Visual Performance,” Technical Report No. UMTRI-97-31, University of Michigan Transportation Research Institute, Ann Arbor, Mich., July 1997, 22 pgs.; and Hornyak et al., “Introduction to Nanoscience,” 2008, CRC Press, Boca Raton, Fla.)

Placing a medical device (e.g., an implantable medical device) inside the body may place a patient at risk for infection and/or allergic reaction. Given the nature of processing medical device components, surface energy may be relatively high, which may allow one or more bodily fluids to wet on them. Wetting of a medical device component may cause, for example, bacterial cultivation and may lead to infection. Another area that has been a concern is allergic reactions. Many alloys utilized in implantable devices have the possibility of causing some sort of inflammatory response. Although this is generally patient dependent, reports of allergic reactions are associated with implant materials. Improved medical devices and medical device components are desired.

Electrocautery grounding pads have been used in the medical device field. Electrocautery grounding pads have included an outer ring of adhesive and in inner area having a sticky conductive gel applied to one of the surfaces of the pad for attachment to a patient. During, for example, a high power application of RF current, such as RFA devices, four grounding pads have been used. Using a plurality of grounding pads may prevent burns to the patient by increasing the grounding surface area and reducing the current density at any specific location on the patient's skin. In some circumstances, the leading edge of a single pad may cause a burn or a wrinkle in the pad and may result in a high current density at that location on the return path. Some wrinkles are noticed only during or after the application of the pad (e.g., adherence to the patient's skin) when pads may be difficult to remove or readjust (e.g., without causing patient discomfort, etc.). Inadvertent trapping of air (e.g., between the electrocautery grounding pad and a patient's skin) may cause the tissue contact to be irregular, which may cause an increase in current density of the return path and may reduce the ability to have a gradient return path to the tissue surface Improved grounding pads are desired.

Research has been conducted in the area of using micropatterned adhesives in wet biological applications. For example, applications of this research include endoscopic robots and biodegradable tissue adhesives. (See, e.g., Lotters et al., “The mechanical properties of the rubber elastic polymer polydimethylsiloxane for sensor applications,” J. Micromech. Microengineering, 1997, 7(3):145-147; Axisa et al., “Low cost, biocompatible elastic and conformable electronic technologies using MID in stretchable polymer,” Conf. Proc. IEEE Eng. Med. Biol. Soc., 2007; 2007:6593-6; Jeong et al., “Nanohairs and nanotubes: Efficient structural elements for gecko-inspired artificial dry adhesives,” Nano Today, August 2009, 4(4):335-346; and Majidi, “Enhanced Friction and Adhesion with Biologically Inspired Fiber Arrays,” University of California, Berkeley, Ph.D. thesis, May 15, 2007, 143 pgs.) Although not wishing to be bound by theory, the mechanism for micropattern attachment to tissue (e.g., the digestive tract) may be based on the ability of the tissue to conform to the micropatterned surface and interlock with it in these applications. As a result, architectures have evolved to less-closely resemble the hair-like structures found on the feet of a gecko. For example, although not wishing to be bound by theory, by decreasing pillar density and aspect ratio, it is possible to achieve greater pillar-tissue interlock. (See, e.g., Mandavi et al., “A biodegradable and biocompatible gecko-inspired tissue adhesive,” Proc. Natl. Acad. Sci. U.S.A., 2008 Feb. 19; 105(7):2307-12 (see, e.g., FIG. 4f at pg. 2311)).

The following documents relate to techniques for manufacturing a micropatterned surface, each of which is incorporated by reference in its entirety: Kroetch, “NanoFab's PDMS Microfluidic Device Fabrication Manual,” September 2004, 8 pgs. (available online at http://www.nanofab.ualberta.ca/wp-content/uploads/2009/03/boxedpdms.pdf, last accessed Mar. 10, 2013); Dodou et al., “Mucoadhesive micropatterns for enhanced grip,” Conf. Proc. IEEE Eng. Med. Biol. Soc., 2007; 2007:1457-62; Kwon et al., “Friction enhancement via micro-patterned wet elastomer adhesives on small intestinal surfaces,” Biomed. Mater., 2006 December; 1(4):216-20; Tooley et al., “Thermal fracture of oxidized polydimethylsiloxane during soft lithography of nanopost arrays,” J. Micromech. Microeng., 2011, 21:054013 (9 pgs.); and Desai et al., “Plastic masters-rigid templates for soft lithography,” Lab Chip, 2009 Jun. 7; 9(11):1631-7.

Micropillars have been fabricated using a variety of polymeric materials. Indeed, any polymeric material may be used to create a micropatterned adhesive provided that it is flexible enough to conform to the target tissue type and create and effective interlock. (See, e.g., Majidi, “Enhanced Friction and Adhesion with Biologically Inspired Fiber Arrays,” University of California, Berkeley, Ph.D. thesis, May 15, 2007, 143 pgs.)

Although not wishing to be bound by theory, it is believed that cells such as fibroblasts, endothelial cells, and muscle cells actively sense both the external loading applied to them (outside-in signaling) and the stiffness of their surroundings (inside-out signaling) and respond to these stimuli with changes in adhesion, proliferation, locomotion, morphology, and synthetic profile. More details regarding this are provided by Throm Quinlan et al., “Combining dynamic stretch and tunable stiffness to probe cell mechanobiology in vitro,” PLoS One, 2011; 6(8):e23272, which is incorporated herein by reference in its entirety. Also incorporated by reference in its entirety is Yoon et al., “Passive control of cell locomotion using micropatterns: the effect of micropattern geometry on the migratory behavior of adherent cells,” Lab Chip, 2012 Jul. 7; 12(13):2391-2402, which indicates that the amount and gradient of physical spatial cues imposed by changing the width and divergence angle of micropatterns make it possible to control the rate and direction of cell migration in a passive way, the results of which offer a potential for reducing the healing time of open wounds.

For example, a graphical representation of the cellular characteristics of the wound healing process is presented by de la Torre et al. (de la Torre et al., “Chronic Wounds,” MedScape Reference—Drugs, Diseases & Procedures, available at http://emedicine.medscape.com/article/1298452-overview#showall) (last accessed Mar. 7, 2013)), incorporated by reference in its entirety, including different types of cell involvement over the course of wound healing. The progression of specific cell, matrix, or processes each maximize in the following order according to de la Torre et al.: platelets, neutrophilia, macrophagen, lymphocytes, capillaries & epithelium, fibroblasts, and collagen. De la Torre indicates that in the second stage of the inflammatory phase, leukocytes supplant platelets as the dominant cell type, attracted by chemotaxis (chemical signaling through growth factor/protein concentrations). White blood cells are the predominant cells for the first 3 days after wounding and after 24-36 hours, circulating monocytes enter the wound and mature into tissue macrophages. These cells debride the wound on the microscopic level and produce a wide variety of important substances, such as IL-1 and basic fibroblast growth factor (bFGF). IL-1 stimulates the proliferation of inflammatory cells and promotes angiogenesis through endothelial cell replications. bFGF is a chemotactic and mitogenic factor for fibroblasts and endothelial cells. Two to three days after healing, fibroblasts migrate inward from wound margins over the fibrinous matrix established during the inflammatory phase. During the first week, fibroblasts begin producing glycosaminoglycans and proteoglycans, the ground substance for granulation tissue, as well as collagen, in response to macrophage-synthesized bFGF and TGF-β, as well as PDGF (growth factors that influence cell behavior).

### DETAILED DESCRIPTION

While the subject matter of the present disclosure may be embodied in many different forms, there are described in detail herein specific preferred embodiments of the present disclosure. This description is an exemplification of the principles of the present disclosure and is not intended to limit the present disclosure to the particular embodiments illustrated.

For the purposes of this disclosure, like reference numerals in the figures shall refer to like features unless otherwise indicated.

The present disclosure relates to employing a micropatterned polymeric coating for, for example, the reduction of friction between two parts of a device in sliding contact or between a part of the device and tissue in sliding contact. In one or more embodiments, the micropatterned polymeric coating may provide a coated device or portion thereof with a non-stick characteristic, wherein articles that contact the non-stick surface may slide along the non-stick surface more easily and/or may be more easily and more completely removed from the non-stick surface.

It should be noted that micropatterned polymer coating, as used herein, may refer to a separately manufactured polymer material that is applied to a surface or may refer to a polymer material that is manufactured simultaneously with the surface or may refer to a medical device surface having a micropattern incorporated thereon. Further, micropatterned polymer coating may be formed of any suitable material for a particular application, and may include one or more of a flexible polymer, a rigid polymer, a metal, an alloy, and any other material that may be suitable for a particular application.

For this disclosure, where reference is made to a polymeric surface coating with a micropattern, it is understood that the pattern can also be applied to a surface lacking a coating.

Biopsy Cap Having Micropattern

As shown, for example, in FIG. 1, in one or more embodiments of the present disclosure, a medical device (e.g., a biopsy cap) may have a micropatterned surface (e.g., polymer coating) thereon. There exists an ongoing need for improved biopsy caps.

In one or more embodiments, a micropatterned polymer coating may be applied to a biopsy cap 10 (e.g., the entrance and exit sealing surfaces). For example, an entry or exit surface component (e.g., entry web 11) could take the form of any of a multitude of geometries while incorporating a lubricious texture pattern (having a micropattern surface 12), which may thereby reduce the frictional forces required for devices to enter and exit a scope through the biopsy cap.

For example, in FIG. 1, a biopsy cap 10 is shown wherein the biopsy cap entry web 11 could be a separate component with desirable lubricious features built into it and then attached to the cap 10 or incorporated into the cap 10 itself. The shape of the biopsy cap entry web 11 may be varied to improve the sealing nature of the septum with the additional surface features (e.g., micropillars of a micropatterned polymer coating) incorporated to reduce frictional forces. In one or more embodiments, a predefined texture pattern may be incorporated into a sealing septum that may, for example, aid in the adhesion of a lubricious coating (e.g., a micropatterned polymer coating) that could be added to the septum after manufacture.

Non-Fouling/Antibacterial/Hypoallergenic Micropatterned Surfaces

As shown, for example, in FIG. 4, in one or more embodiments of the present disclosure, a medical device (e.g., a lens) may have a micropatterned surface (e.g., polymer coating) thereon. More broadly, there exists an ongoing need for improved medical device surfaces that are, for example, non-fouling, antibacterial, and/or hypoallergenic.

In one or more embodiments, a micropatterned polymer coating may provide a non-fouling surface, antibacterial surface, and/or hypoallergenic surface, which may be useful in a wide variety of devices (e.g., medical devices).

For example, in one or more embodiments, as shown in FIG. 4, a medical device may include a substrate, such as a lens 25, having a micropatterned surface 26 thereon, wherein the micropattern surface includes micropattern features (e.g., a micropatterned polymer coating) to create one or more superhydrophobic surfaces that repel fluids, which may reduce the need for frequent water flushing to remove debris.

Thus, use of a micropatterned polymer surface or coating may provide, in one or more embodiments, a non-fouling lens material or a coating for a traditional endoscope and smaller imaging systems that allow for direct visualization of, for example, the biliary system. The gastrointestinal tract and ducts of the biliary system are generally covered with fluids such as stomach acid, blood, mucous, and bile. These fluids can create a challenging environment for optical imaging systems due to their masking effect when they contaminate an imaging lens. Endoscopic imaging systems have attempted to reduce the impact of body fluids by including a water-flush system to clean the lens. The wash systems often require frequent activation to clear the lens, which slows the procedure and adds significant amounts of water to the viewing area.

Superhydrophobic surfaces (e.g., such as a micropatterned surface 26) created by patterning the lens surface (e.g., lens 25) may prevent lens fouling with reduced water flushing. In at least one embodiment, droplets of liquid contact the lens 25, are repelled by the micropattern surface 26, and drip off the lens 25, which may allow for clean visualization. In one or more embodiments, the lens may be applied to a SPYSCOPE® (commercially available from Boston Scientific Scimed, Inc.), a traditional scope, or an endoscope cap.

As shown in the schematic of FIG. 2, the micropattern may be provided as a plurality of nanostructures 14, in which a plurality of protrusions (e.g., pillars) have a nano-scale (e.g., have at least one dimension selected from height and width that is less than 1000 nanometers). In one or more embodiments, a micropattern may be provided as a plurality of microstructures 15, in which a plurality of protrusions have a micro-scale (e.g., have height and width dimensions of 1 micrometer or greater). In one or more embodiments, a micropattern may be provided as a plurality of hierarchical structures 16, in which a plurality of protrusions having a nano-scale 17 are combined with a plurality of protrusions having a micro-scale 18. As shown in FIG. 2, a hierarchical structure 16 may include nano-scale protrusions (e.g., nano-scale pillars) disposed on one or more of a plurality of protrusions having a micro-scale (e.g., micro-pillars). Although not wishing to be bound by theory, it is believed that adjusting the dimensions of the protrusions (e.g., pillars) and the arrangement of and dimensions between the protrusions will affect, for example, how a droplet of a particular liquid 19 will engage the surface 20.

Transparent glass surfaces have been rendered superhydrophobic with thin films. See, e.g., Su et al., “Fabrication of an optically transparent super-hydrophobic surface via embedding nano-silica,” Applied Surface Science, 30 Dec. 2006, 253(5):2633-2636, incorporated herein by reference in its entirety. Other superhydrophobic surfaces have been developed. See, e.g., “Superhydrophobe,” Wikimedia Foundation, Inc., 2013 (available on the internet at http://en.wikipedia.org/wiki/Superhydrophobe) (last accessed Mar. 15, 2013).

In one or more embodiments, a micropattern may be incorporated directly into a lens 25 material, thereby forming a micropatterned surface 26. Such an incorporated micropatterned surface may be useful in that it may be more durable than a soft polymer coating having a micropatterned surface and may be fabricated without any additional materials that may present issues regarding device compatibility. Thus, in one or more embodiments, a micropatterned surface may be incorporated into any of a wide variety of lens materials. In some embodiments, a micropatterned surface may be incorporated more broadly into other glass hardware that contact one or more liquids.

As described above, properly dimensioned patterned (e.g., micropatterned) surfaces may render a material superhydrophobic and/or fluid repellent. Such characteristics may be measured by, for example, measuring a contact angle formed by a droplet of water on the surface. As shown in FIG. 3, generally a droplet of liquid 19 will contact a solid 20 (e.g., a base) and form a contact angle (e.g., 0). This contact angle may be modified by modifying the structure of the surface contacted by the droplet. At least two theoretical models have been used to describe the wetting regimes, including the Wenzel model which describes a homogeneous wetting regime as shown in FIG. 4 and the Cassie-Baxter model which describes a heterogeneous surface, such as those shown in FIG. 3.

Marking Device Having Micropattern

As shown, for example, in FIG. 5, in one or more embodiments of the present disclosure, a medical device (e.g., a marking device) may have a micropatterned surface (e.g., polymer coating) thereon. There exists an ongoing need for improved marking devices.

In one or more embodiments, a micropatterned surface (e.g., a micropatterned polymer coating) may be applied to a marking device. For example, a marking device 28 as depicted in FIG. 5 may include a marker portion 29 (e.g., a biomarker) and a micropatterned polymer coating 30 (e.g., having an adhesive texture) attached to the marker portion 29, wherein the micropatterned polymer coating 30 includes adhesive qualities structured and arranged to adhere to biological tissue and/or an implantable medical device.

In one or more embodiments, the micropatterned polymer coating 30 may be attached to the marker portion 29 in any of a wide variety of ways known to one of skill in the art including, but not limited to, adhered, bonded, mechanically coupled, sutured, etc. In at least one embodiment, marking portion 29 may take any of a wide variety of geometries, depending on the anatomy in which the marking device 28 will be disposed and depending on the geometries of the delivery system. Marking portion 29 may include or be made from one or more radiopaque materials, which can, for example, make the marking portion visible under fluoroscopy. In at least one embodiment, micropatterned polymer coating 30 may be manufactured separately from the marker portion 29 and later attached to the marker portion 29 (e.g., by adhesive, etc.).

Alternatively, a micropatterned surface 31 (FIG. 6) may be incorporated into the marker portion 29 (e.g., by etching, etc.) itself.

In one or more embodiments, marking device 28 may be implanted into a patient, wherein marking device 28 remains in place due to the adhesive quality of the micropatterned surface (e.g., micropatterned polymer coating 30). In one or more embodiments, the marker portion 29 may be resorbable. In one or more embodiments, the micropatterned polymer coating 30 may be designed to adhere the marker portion 29 in place for a pre-determined amount of time.

In one or more embodiments, use of a micropatterned polymer coating 30 in a marking device 28 may offer one or more advantages, such as reducing or eliminating the need to use conventional clipping methods to hold a marking device 28 in position, being less invasive, and the ability to be custom designed based on the anatomical location targeted. A marking device 28 of the present disclosure may be useful in placing indicators for review and/or for placement of additional devices.

Wound Patch System Having Micropattern

As shown, for example, in FIG. 7, in one or more embodiments of the present disclosure, a medical device (e.g., a wound patch system) may have a micropatterned surface (e.g., polymer coating) thereon. There exists an ongoing need for improved devices and methods for healing wounds.

An adhesive micropatterned polymer coating may also be useful in treating wounds. For example, referring to FIG. 7, a wound patch system can include one or more pads 36 (e.g., a wound patch), each of which may include an adhesive micropattern surface (e.g., a micropatterned polymer coating) for adhering to tissue 37 at or near a wound site 38, wherein the micropatterned surface is oriented on a pad 36 to contact tissue 37.

In one or more embodiments, a wound patch system 35 may include a second micropatterned surface (e.g., micropatterned polymer coating), wherein the micropatterned surface is structured and arranged to enhance wound healing. In at least one embodiment, a wound patch 36 having an adhesive micropatterned polymer coating may be deployed over a wound, wherein an adhesive micropatterned polymer coating adheres to tissue near a wound side and optionally includes a second micropattern designed for enhancing wound healing. Further information regarding micropatterns disposed directly over a wound site may be found in U.S. Pat. App. Ser. No. 61/798,685 (Firstenberg et al.), entitled ANTI-MIGRATION MICROPATTERNED STENT COATING, filed on the same date herewith (Atty. Docket No. S63.2-15576US02).

In at least one embodiment, each of a plurality of patches 36 may include an adhesive micropatterned polymer coating for adhering to tissue 37 near a wound site 38 and further being structured and arranged to be manipulated by, for example, a suture in order to pull two or more wound patches 36 closer together, effectively pulling two or more sides of a wound closer together (e.g., to close the wound). The plurality of patches 36 may then be positionally stabilized in any manner (e.g., fixated by a larger patch, fixated by suture, adhered, bonded, sewn, etc.) so that the wound may remain closed.

FIG. 7 depicts one exemplary system 35 including a plurality of pads (e.g., two pads) 36, each of which includes a micropatterned polymer coating constructed and arranged to adhere to tissue 37 (e.g., near a wound site 38). The system 35 also includes a suture 39 (e.g., a filament, thread, wire, etc.) threaded through at least a portion of at least two of the pads 36 such that a proximally directed force exerted on the suture 39 (or suture system) may be translated into a force on each pad 36 drawing it close to the another pad 36 (e.g., drawing the pads closer together). The system 35 may optionally include a fixation mechanism to maintain the distance between the two pads (e.g., to secure the relative pad position after being brought closer together).

Tools Having Micropattern for Gripping Tissue

As shown, for example, in FIG. 8, in one or more embodiments of the present disclosure, a medical device (e.g., a hemostasis clip, biopsy forceps, etc.) may have a micropatterned surface (e.g., polymer coating) thereon. There exists an ongoing need for improved medical devices for gripping biological tissue.

In one or more embodiments, an adhesive micropatterned polymer surface (e.g., a micropatterned polymer coating) may be used in applications in which biological tissue is to be retained (e.g., in a hemostasis clip, etc.).

For example, as shown in FIG. 8, a hemostatis clip 40 may include a micropatterned polymer coating 41 on one or more surfaces on the inside of one or more jaws 42 of the hemostasis clip 40, which may adhere to tissue before and/or during the clip is deployed. The inclusion of a micropatterned polymer coating 41 on one or more inside surfaces of a hemostasis clip 40 may allow more accurate deployment of the hemostasis clip 40 and more reliable closing of a bleed, for example.

In one or more embodiments, a hemostasis clip 40 may include a lubricious micropatterned polymer coating on, for example, the outside surface 44 of a hemostasis clip 40 (e.g., one or more outer-facing surfaces 44 of each jaw 42 of a hemostasis clip 40) (e.g., FIG. 9) where the outer surface 44 may contact a capsule 43 during the closing of the hemostatis clip jaws 42. One or more of the jaws 42 may ride on and move inside of the capsule 43, wherein the sliding of the contacting (e.g., mating) surfaces is obstructed by friction. Friction between these sliding surfaces may cause a need for higher deployment and retraction forces, which may be reduced by applying a lubricious micropatterned polymer coating on one or both of the outside surface of the jaws 42 and an inside surface 45 of the capsule 43. In one or more embodiments, reducing the frictional forces to be overcome during deployment and/or retraction with, for example a micropatterned polymer coating, may allow for more controlled handling and more accurate placement.

In one or more embodiments, a micropatterned surface (e.g., a micropatterned polymeric coating) may be applied to (or incorporated within) a medical device including a biopsy forceps. An exemplary biopsy forceps tool 46 is shown in FIG. 10, wherein the biopsy forceps 46 includes two jaws 47, each of which includes a concave shape and a cutting edge that can mate with a cutting edge 48 of the other jaw. As shown in FIG. 10, an inner surface of a jaw may include a micropatterned surface (e.g., a micropatterned polymer coating). For example, additional details regarding one or more micropatterned surfaces applied to (or incorporated within) a biopsy forceps tool are provided by U.S. Pat. App. Ser. No. 61/798,991 (Bertolino et al.), entitled BIOPSY TOOL HAVING MICROPATTERN, filed on the same date herewith (Atty. Docket No. S63.2-15935US01) and the documents cited therein.

Tools Having Micropattern

As shown, for example, in FIG. 11, in one or more embodiments of the present disclosure, a medical device or a portion thereof (e.g., handle, a knob, a glove, a guidewire, a snare, etc.) may have a micropatterned surface (e.g., polymer coating) thereon. There exists an ongoing need for improved medical devices having improved surfaces thereon in order to, for example, improve handling.

In one or more embodiments, a micropatterned surface (e.g., a micropatterned polymer coating) having increased adhesive or friction qualities may be applied to or incorporated into a torque device associated with manipulating a guidewire or to a tool that is used to rotate, push, or pull a guidewire. As shown in FIG. 13, in one or more embodiments, for example, a micropatterned polymer coating may be applied or incorporated into, for example, a surgical glove 50 wherein the portions of the glove that may be used to grip a guidewire (e.g., near fingertips 51 of glove, 50, on or near palm 52 of glove 50, etc.) have increased adhesive and/or frictional characteristics. A micropatterned polymer coating may be applied to a wide variety of gloves (e.g., surgical gloves) for use in a wide variety of applications (e.g., surgical procedures, endoscopic procedures, etc.).

In one or more embodiments, a micropatterned surface (e.g., micropatterned polymer coating) may be constructed and arranged to provide a non-slip surface (e.g., having increased frictional qualities and/or increased adhesive qualities) on, for example, a tool 55 or a portion thereof. Referring to FIG. 11 and FIG. 12, for example, a non-slip surface may be useful on portions of tools 55 where the tool 55 is handled (e.g., a handle 56, a grip 56a, etc.), on buttons, wheels 57, on knobs 58, and/or on surfaces that may grip the tool (e.g., portions of a surgical glove, etc.). In one or more embodiments, a non-slip surface provided by a micropatterned polymer surface may provide increased security grip for applications in which improved control, torque, tactile feel, and tactile transfer are desired.

In FIG. 14, a schematic is provided, showing a micropatterned surface 60 in a conformal engagement with another surface 61 (e.g., biological tissue, another micropatterned surface, etc.). As shown, the micropatterned surface 60 includes a plurality of protrusions 62 (e.g., micropillars). Additional information regarding micropatterns and micropillars is provided in the applications listed on page 1 and the documents cited therein, all of which are incorporated herein by reference, each in its entirety.

In one or more embodiments, a micropatterned polymer coating may improve the ergonomic qualities of a medical device or tool 55. In one or more embodiments, a micropatterned surface (e.g., micropatterned polymer coating) may be applied to both surfaces that may mate during use of a tool (e.g., a micropatterned handle with a micropatterned glove on a hand gripping the handle, etc.). In some embodiments, a micropatterned polymer coating that provides a non-slip surface may allow application of a higher torque control with reduced corresponding grip force.

In one or more embodiments, a region of a tool 50 (e.g., a handle) may include one or more non-slip surfaces that may be continuous (see, e.g., FIG. 11) or may not be continuous (see, e.g., FIG. 12). The plurality of non-slip surfaces may be strategically placed on the handle in order to provide an adequate level of increased control while reducing the quantity of micropatterned polymer coating.

In one or more embodiments, a micropatterned polymer coating may be applied to a tool part or feature by a secondary process such as laser etching, material transfer, a paint or spray or dipping process, a pre-made film that is bonded to the tool part, or by any other suitable means known to one of skill in the art.

There exist many useful locations on tools for micropattern surfaces to be applied in order to improve handling of tools and/or medical devices. Details regarding human factors and ergonomics for handles, hand grips, knobs, grip strength, and other topics may be found in one or more of the following documents, each of which is incorporated by reference in its entirety: Woodson et al., “Human Factors Design Handbook: Information and Guidelines for the Design of Systems, Facilities, Equipment, and Products for Human Use,” 2nd ed., New York, N.Y., McGraw-Hill, 1992; Greig et al. “Measurement of Prehensile Grasp Capabilities by a Force and Moment Wrench: Methodological Development and Assessment of Manual Workers,” Ergonomics, 2004 Jan. 15; 47(1):41-58; Department of Defense, Human Engineering Design Data Digest, April 2000 (151 pages) (available at http://www.dtic.mil/cgi-bin/GetTRDoc?AD=ADA467401) (last accessed Mar. 8, 2013).

In one or more embodiments, selecting a particular micropattern (e.g., the geometric arrangement of micropillars in a micropatterned surface, the micropillar dimensions, etc.) for a given application may allow improved frictional characteristics.

Use of guidewire polymer jacket may benefit, in some embodiments, from a micropattern having rows of micropillars arranged in the direction of the longitudinal axis of the guidewire. For example, in FIG. 16, a schematic of a longitudinal micropattern 66 (e.g., micropillars arranged in rows parallel to a longitudinal axis of the guidewire 65) is shown on a guidewire 65, which may reduce the push force necessary to push a guidewire 65. In another example, shown in FIG. 15, a schematic of a micropattern 68 having helical rows of micropillars on a guidewire jacket 67 may reduce the force required for rotating a guidewire. In one or more embodiments, a stylet with a longitudinal micropattern 70 may require a reduced push force. In some embodiments, a guidewire polymer jacket 67 including a longitudinal micropattern may require a reduced push force.

Two common guidewire jacket materials are PTFE and urethane. In one or more embodiments, a micropattern will include longitudinal and circumferential features to facilitate axial and rotational motion of the wire (e.g., within a lumen). In one or more embodiments, a micropattern may be applied to an extruded polymer tube before applying the tube to a core wire (e.g., by shrinking). In some embodiments, the micropattern may be applied to a guidewire jacket after it is attached to the core wire using, for example, a roll forming or coating process.

In one or more embodiments, a proximal end friction increasing micropattern may be applied to a separate device, such as a finger cot or glove. The increased-friction finger cot may be supplied with the guidewire and worn by an operator manipulating the wire, which may allow for improved control of the wire.

Guidewires Having Micropatterns

As shown, for example, in FIG. 17, in one or more embodiments of the present disclosure, a medical device or a portion thereof (e.g., guidewire) may have a micropatterned surface (e.g., polymer coating) thereon. There exists an ongoing need for improved guidewires having improved surfaces thereon in order to, for example, improve handling.

With reference to FIG. 17, in one or more embodiments, a micropatterned surface (e.g., micropatterned polymer coating 75) may be applied to a guidewire 74 wherein the micropatterned polymer coating is disposed on the outside and along the length of the guidewire or a portion thereof (e.g., to provide increased surface friction). For example, a micropatterned surface providing increased friction may be applied to a portion (e.g., proximal portion 76) of a guidewire 74 handled by an operator (e.g., a proximal portion at or near the proximal end 77 of the guidewire 74).

In one or more embodiments, a friction-increasing micropattern may be applied to a distal portion 78 (e.g., the distal tip 79) of the guidewire 74 in order to, for example, improve distal tip anchoring in biological tissue (e.g., an organ). For example, in at least one embodiment, a guidewire 74 may include a micropatterned portion at or near the distal end 79 of the guidewire, wherein the micropatterned portion may stick to (e.g., adhere, be friction fitted with, etc.) a portion of a gastrointestinal wall (not shown) beyond a stricture to aid in holding the guidewire 74 in place during, for example, a stent placement (e.g., delivery, deployment, etc.), which may avoid the need to advance and detract the guidewire 74 during the stent-delivery procedure and may reduce miscommunication between personnel (e.g., doctors, nurses, etc.) involved with the procedure.

In one or more embodiments, a friction-reducing micropattern surface (e.g., micropatterned polymer coating) may be present on a guidewire, which can allow easier insertion, rotation, and stricture passage.

In one or more embodiments, friction-increasing micropatterns may be used separately from or in combination with a friction-reducing micropattern. Modifying various structural characteristics of a micropattern (e.g., micropillar dimensions, micropillar area density, micropillar material, etc.) can have an effect on the lubricity and/or frictional characteristics of a micropatterned polymer coating.

Medical guidewires have been used in combination with a catheter, tome, or cannula and may traverse tortuous paths before reaching a desired treatment location, thereby providing a route for other medical devices to follow. Increased friction between a guidewire and a supporting device may reduce the feel and response of the wire for an operator (e.g., a physician). For example, it has been reported that a wire may bind in the tome or cannula and prevent further insertion or may hinder the ability to rotate the guidewire tip. Hydrophilic coatings have been applied to guidewires to help reduce friction, but such coatings must remain wet to reduce friction and may be slippery for an operator to handle on the proximal end of a guidewire.

Referring FIG. 18, in one or more embodiments, a distal tip 80 of a guidewire 81 may include a lubricious micropatterned polymer coating 83 that extends only partially circumferentially around the guidewire and a friction-increasing micropatterned polymer coating 82 that extends only partially circumferentially around the guidewire. In some embodiments, a lubricious tip may allow for easier advancement through a tortuous body lumen 84 and may be combined with a friction-increasing micropatterned polymer coating that extends entirely circumferentially around the guidewire along a longitudinal portion of the guidewire that is separated from (e.g., near to) the distal tip.

With reference to FIG. 19, a guidewire 85, in some embodiments, may include a micropatterned surface 86 (e.g., a micropatterned polymer coating) over a longitudinal portion of the guidewire 85. In FIG. 19, the micropatterned surface extends from a guidewire tip 87. In some embodiments, a micropatterned surface 86 extends along a longitudinal portion of a guidewire that does not include a tip.

Medical Devices Having Micropattern for Reduced Friction

As shown, for example, in FIG. 21, in one or more embodiments of the present disclosure, a medical device or a portion thereof (e.g., needle, catheter, etc.) may have a micropatterned surface (e.g., polymer coating) thereon. There exists an ongoing need for improved medical devices.

For example, in one embodiment, a micropatterned polymeric coating may be applied to an FNA (fine needle aspiration) needle. In at least one embodiment, with reference to FIG. 21, use of a micropatterned polymeric coating 96 on a needle tip 97 may reduce the force necessary to puncture tissue. In at least one embodiment, use of a micropatterned polymeric coating on the outside of the needle wall may reduce the force necessary to slide the needle through the puncture. In at least one embodiment, use of a micropatterned polymeric coating on the inside of the needle wall may reduce the force necessary to obtain a biological tissue sample and to release the biological tissue sample after the procedure is complete.

In one or more embodiments, a micropatterned polymeric coating may be applied to (or incorporated within) a medical device including an atraumatic quill.

In one or more embodiments, a micropatterned polymeric coating may be applied to sclerotherapy needles (e.g., Interject® needles from Boston Scientific) used in, for example, injection therapy applications. Sclerotherapy needles may be used in tortuous anatomy and may benefit from a strong-pushable sheath construction for advancement through difficult pathways and remote anatomy. Due to surface friction and the tortuosity of the paths in sclerotherapy, precise positioning of the sclerotherapy needles may be difficult to achieve and/or difficult to maintain during the procedure. Improved precision of movements of a sclerotherapy needle within, for example, a sheath construction may be a benefit of applying a micropatterned polymeric coating to either the outer surface of the sclerotherapy needle or the inside surface of the sheath in which the needle slides, or both. In at least one embodiment, referring to FIG. 21, a surface of a sheath 99 may have a micropatterned surface 98 (e.g., micropatterned polymer coating) thereon to allow for increased control and precise movement of the needle 97a therein and needle tip 97 extending therefrom.

In one or more embodiments, referring to FIG. 24, a micropatterned polymer coating (e.g., a Hoowaki pattern (see http:www.hoowaki.com)) may be applied to a medical delivery device (e.g., an endoscopic delivery device) including a tube 300 wherein the micropatterned polymer coating 301 is disposed on the inside of the tube (e.g., to reduce deployment force) and another micropatterned polymer coating 302 on the outside of the tube 300 (e.g., to facilitate introduction of the tube within biological tissue such as a body lumen).

In one or more embodiments, a micropatterned polymer coating 306 may be applied to a guidewire 305 (e.g., FIG. 25) wherein the micropatterned polymer coating is disposed on the outside and along the length of the guidewire or a portion thereof (e.g., to facilitate sliding the guidewire within another medical device or component thereof or within a body lumen). A guidewire with increased lubricity would be the result. In one or more embodiments, a guidewire may include a friction-reducing micropattern polymer coating along at least a portion of a guidewire (e.g., along the entire length of the guidewire) to allow for, for example, easy insertion, rotation, and stricture passage without the need for additional surface coatings.

In one or more embodiments, a micropatterned polymer coating may be applied to the tip of the sclerotheraphy needle, wherein the micropatterned polymer coating may provide increased ease of insertion when puncturing biological tissue.

In one or more embodiments of the present disclosure, a micropatterned polymer coating may provide an increased lubricity to a medical device or portion thereof.

In one or more embodiments, a micropatterned polymer coating may be applied to a medical device in which a portion thereof is subjected to torsional rotation (e.g., within a sheath). For example, with reference to FIG. 23, snares 310 (e.g., flat-wire snares) and other devices that provide stiffness have been known to experience difficulty in torsional rotation or flipping within an endoscopic device 311 (e.g., a tube, a sheath, etc.). In one or more embodiments, a micropatterned polymer coating may be applied to a medical device portion subjected to torsional rotation (e.g., a snare, a guidewire, etc.) such as a snare 310 or to a portion 312 of the medical device within which the other portion (e.g., the snare 310) rotates, or both portions (e.g., two contacting surfaces having micropatterned polymer coatings). In one or more embodiments, the use of micropatterned polymer coatings may provide for delamination/isolation of two portions of a medical device.

In one or more embodiments, a micropatterned polymer coating may be applied in addition to or as an alternative to one or more conventional lubricants such as MDX (e.g., MDX4-4159 50% medical grade dispersion (commercially available from Dow Corning Co.), which has been used in medical applications including siliconization, hydrophobing, and lubrication of syringes, needles, vials, stoppers, etc.)). For example, some conventional lubricants (e.g., MDX), when used on various medical devices or products such as biopsy forceps, may become tacky when applied incorrectly. A micropatterned polymeric coating may be applied to a surface to either bind layers together (e.g., increase tackiness) or to reduce friction between portions that are in sliding contact. For example, with reference to FIG. 20, in one or more embodiments, a micropatterned polymeric coating 90 may be applied to control wires 91 used in, for example, endoscopic procedures. The micropatterned polymeric coating may reduce the friction associated with sliding the wires 91 through channels 92 within a catheter 93, a sheath, or other endoscopic tube.

As described elsewhere herein, in one or more embodiments, a micropatterned polymer coating may provide increased adhesion or tackiness to a medical device surface or portion thereof. For example, as shown in FIG. 22, a stent delivery device 94 may be improved by applying a micropatterned polymer coating 95 to the stent-holding portion 89 of a stent delivery device, in order to more reliably hold a stent within the device prior to deployment.

Grounding Pads Having Micropattern

As shown, for example, in FIG. 27, in one or more embodiments of the present disclosure, a medical device (e.g., an electrocautery grounding pad, etc.) may have a micropatterned surface (e.g., polymer coating) thereon. There exists an ongoing need for improved electrocautery grounding pads.

With reference to FIG. 27, in one or more embodiments, a micropatterned polymer coating 315 may be applied to an electrocautery grounding pad 316 (e.g., an RF grounding pad). For example, a micropatterned polymer coating may be applied to that it is tacky in order to adhere to tissue and yet have characteristics that may allow it to be removed when peeled in a specific manner (e.g., in a specific direction, with a change in pressure applied, etc.). Use of a micropatterned polymer coating to provide an adhesive surface may allow the reduction or elimination of the use of sticky gels that have been associated with use of electrocautery grounding pads. Such gels have tended to adhere to a patient and have been difficult to remove from the patient's skin after use of an electrocautery grounding pad.

In one or more embodiments, the micropatterned polymer coating may include projections that are angled from their origin surface (see angled microfeatures 316 in FIG. 26). In one or more embodiments, the micropattern may be applied in an array pattern to encourage the outer edges of the electrocautery grounding pad to adhere to a patient's skin.

In one or more embodiments, a micropatterned polymer coating may be applied to all or a portion of an electrocautery grounding pad surface. For example, a micropatterned polymer coating may entirely cover a surface of the electrocautery grounding pad surface. A micropatterned polymer coating may, in some embodiments, be combined with a different adhesive arranged around the perimeter of the pad. In still other embodiments, a micropatterned polymer coating may be combined with a different adhesive, wherein the micropatterned polymer coating is arranged around the perimeter of the pad.

In one or more embodiments, with reference to FIG. 26, a micropatterned polymer coating may be structured and arranged to include microfeatures 320 (e.g., micropillars) to increase the surface contact to tissue 321 in order to, for example, encourage a gradient return path to prevent burns thereby not allowing areas of high current density to occur at any specific location on a skin surface.

In one or more embodiments, a micropatterned polymer coating may be hydrophilic and/or include a conductive material in a suitable concentration (e.g., for micropatterns created using otherwise non-conductive materials).

Gastrostomy and Enteral Medical Devices Having Micropatterns

FIG. 28 shows a medical device (e.g., enteral feeding device, etc.) that may include a micropatterned surface (e.g., polymer coating) thereon on one or more of its components, as described herein with reference to FIGS. 29 through 36 and 38 through 41. As shown, for example, in FIG. 29, in one or more embodiments of the present disclosure, a medical device (e.g., enteral feeding device, etc.) may have a micropatterned surface (e.g., polymer coating) thereon. There exists an ongoing need for improved gastrostomy and enteral feeding devices (e.g., percutaneous endoscopic gastrostomy devices, etc.).

In one or more embodiments, a medical device may include one or more portions having a micropatterned polymer coating thereon to provide an adhesive surface and may also include one or more portions having a micropatterned polymer coating thereon to provide lubricious surface.

Enteral feeding devices may provide nutrition to a patient by way of, for example, a feeding tube. In one or more embodiments, an adhesive micropatterned polymer coating may be applied to one or more portions of an enteral feeding device such as a gastrostomy tube system 325 including a balloon 326, a tube 327, a stopper 328, or one or more portions of any of those.

For example, an adhesive micropatterned polymer coating may be disposed on, for example, an external surface of an enteral feeding device that may be constructed and arranged to contact biological tissue within a patient. The adhesive micropatterned polymer coating may be useful in preventing slippage of the enteral feeding device disposed, for example, within a body lumen (e.g., esophagus, intestines, etc.) or body cavity (e.g., nasal sinus, stomach, etc.). In at least one embodiment, an adhesive micropatterned polymer coating 340 may be disposed on at least a portion of a feeding tube, as shown in FIG. 29, and on at least a proximal portion of a balloon 326. In one or more embodiments, an adhesive micropatterned polymer coating may be useful in accessories and replacement products associated with enteral feeding devices, such as the device shown in FIG. 30, wherein a micropatterned polymer coating may be disposed on the surfaces identified in FIG. 31. FIG. 37 also depicts micropatterned polymer coatings on a gastrostomy tube system on, e.g., the balloon 326 and tube 327.

In one or more embodiments, an anti-adhesion and/or anti-microbial micropatterned polymer coating may be applied to one or more surfaces of an enteral feeding device where, for example, a reduction or prevention of adhesion of bacteria or biofilm may be desired. For example, an anti-adhesion or anti-microbial micropatterned polymer coating may be disposed on the wall of an enteral feeding device lumen through which solids, fluids, and/or gases may pass.

For example, an anti-adhesive polymer coating 350 may be applied to the enteral feeding device 360 of FIG. 30 on the surfaces identified schematically in FIG. 32. In one or more embodiments, an anti-adhesive polymer coating 350 may be applied to the gastrostomy tube device 325 of FIG. 28 on the surfaces identified schematically in FIG. 33.

In one or more embodiments, a micropatterned polymer coating may be applied to provide an adhesive surface and/or a lubricious surface to any of a wide variety of medical devices or portions thereof including, but not limited to, a gastrostomy tube (e.g., FIG. 28), an enteral feeding tube (e.g., FIG. 30), a nasogastric tube, a jejunal feeding tube device 362 (e.g., FIG. 34). In one or more embodiments, a combination of an adhesive micropatterned polymer coating and a lubricious micropatterned polymer coating may be used in combination. For example, an adhesive micropatterned polymer coating may be useful in a portion of a medical device that is static and contacts biological tissue, whereas a lubricious micropatterned polymer coating may be useful on a portion of a medical device that contacts biological tissue in a sliding contact.

For example, the exterior surface of an oral, nasal, and/or transoesophageal feeding tube may include a lubricious micropatterned polymer coating to facilitate insertion and/or improve patient comfort, whereas the exterior surface may also include a portion having an adhesive micropatterned polymer coating to facilitate position stability (e.g., fixation) of the device when implanted.

In one or more embodiments, the device of FIG. 28 may have a slippery micropatterned polymer coating 350 disposed on the surfaces identified in FIG. 35. In one or more embodiments, the device of FIG. 30 may have a slippery micropatterned polymer coating 350 disposed on the surfaces identified in FIG. 36.

In at least one embodiment, an implantable balloon may be constructed and arranged such that during delivery in a collapsed state, a portion of the balloon having a lubricious micropatterned polymer coating is exposed, facilitating delivery within a body lumen or cavity. Upon inflation of the implantable balloon, one or more portions of the balloon may be exposed, wherein the one or more portions may have one or more adhesive micropatterned polymer coatings to facilitate fixation of the balloon in the body lumen or cavity.

In one or more embodiments, as shown, for example, in FIG. 38, micro and nano patterns (e.g., a superhydrophobic coating) may be applied to enteral feeding products.

The use of a superhydrophobic coating based off the principles of “the lotus effect” that can be applied to the inside of a PEG balloon, the OD of the shaft inside the PEG balloon and/or the OD of the balloon in the area that comes in contact with tissue.

It may be desired to use an enteral feeding device includes parts that remain implanted in a patient longer than 3 months without replacement.

In one or more embodiments, an adhesive micropatterned polymer coating 370 may be applied to one or more of the following surfaces: the outside diameter the shaft inside of the balloon (e.g., FIG. 38); the inner diameter of the balloon to reduce likelihood of saline leaking while inflated (which would otherwise lead to increased replacement frequency) (e.g., FIG. 39); the inner diameter of the balloon (e.g., FIG. 40); and the outside diameter of the shaft inside of the balloon (e.g., FIG. 41), any of which may reduce or eliminate the likelihood of saline leaking during inflation (which would otherwise lead to increased replacement frequency). In one or more embodiments, the outside diameter of the balloon can be coated with a micro-patterned surface to assist with fixing the balloon to the tissue wall, which can result in less frequent replacement

Inflatable Medical Devices Having Micropattern

As shown, for example, in FIG. 42, in one or more embodiments of the present disclosure, an inflatable medical device (e.g., a dilatation balloon, etc.) may have a micropatterned polymer coating thereon. In one or more embodiments, an inflatable medical device having a micropatterned polymer coating thereon may be useful in a wide variety of applications, including cardiology, pulmonology, urology, gastroenterology, and others.

One of the challenges associated with conventional dilatation balloons during balloon dilatation is keeping the balloon stationary within a body lumen and avoiding the balloon slipping within the lumen. As the balloon is dilated, a conventional dilatation balloon may slip (e.g., the “watermelon seeding” effect), which may result in the intended area to be dilated not being sufficiently dilated and subsequent dilations being required to treat the patient. (See, e.g., Alfonso et al., “Implications of the “watermelon seeding” phenomenon during coronary interventions for in-stent restenosis,” Catheter Cardiovasc Interv. 2005 December; 66(4):521-7.) In cardiology cases, the risk associated with multiple inflations is high. Slippage during endoscopic procedures has occurred in dilatation of strictures in the lower esophageal sphincter, biliary strictures, and in other parts of the body.

For example, use of one or more micropatterns on a balloon surface may be useful to improve balloon positioning stability during dilatation. In one or more embodiments, a micropattern can enhance adhesion of a balloon to a body lumen (e.g., a vessel, an airway, a duct, a tract, a stricture, a lower esophageal sphincter, a biliary stricture, etc.) during inflation of the balloon.

An exemplary balloon having a microstructured polymer coating thereon is depicted in FIG. 42. In one or more embodiments, a balloon 100 can include first and second shoulders 102, 104 near the balloon ends (e.g., cone sections, etc.) with a balloon body 106 disposed therebetween. In FIG. 42, the micropattern polymer coating 110 covers the balloon body 106. In one or more embodiments, a portion of the balloon body can be covered. In some embodiments, the balloon shoulders 102, 104 are not covered by a micropatterned polymer coating, which may be helpful for improved (e.g., clear) endoscopic visualization during dilatation.

In FIG. 43, the micropatterned polymer coating 110 includes a plurality of micropillars 112. As shown in the embodiment shown in FIG. 43, the micropillars can be arranged in one or more longitudinal rows and/or one or more circumferential columns. In one or more embodiments, the arrangement of micropillars 112 on a micropatterned polymer coating may be selected to inhibit unintended axial and/or radial movement of a dilatation balloon when inflated within a body lumen.

Micropatterned polymer coatings that may be useful on a dilatation balloon include, but are not limited to, the micropatterns (e.g., the adhesive micropatterns) described herein and may be formed from the polymer coating materials and by the polymer coating manufacturing techniques described herein.

The addition of a micropattern to a balloon surface can be added to treat strictures for disease states. Balloon dilatation is used in the vascular, pulmonary, urology and gastroenterology. The “watermelon seed” effect is referenced in, e.g., Alfonso et al., “Implications of the “watermelon seeding” phenomenon during coronary interventions for in-stent restenosis,” Catheter Cardiovasc Interv. 2005 December; 66(4):521-7.

In one or more embodiments, a micropatterned polymer coating 110 can be customized to allow one or more portions of the balloon 100 to grip a body lumen (e.g., a vessel, an airway, a duct, a tract, etc.) and allow one or more other portions to slide within a lumen, depending on how the balloon unfolds during dilation. That is, a balloon 100 may be folded for delivery wherein portions of the balloon may be exposed that have a micropatterned polymer coating 110 for enhancing movement through a lumen, whereas the balloon 100 may be inflated at a site for dilatation, thereby exposing other portions of the balloon having a micropatterned polymer coating 110 that has a different micropattern suited for gripping a body lumen.

In one or more embodiments, a micropatterned polymer coating can be applied to an inflation lumen of a balloon catheter to reduce the water contact angle and decrease inflation times.

In one or more embodiments, a micropatterned polymer coating design can be transferred to the balloon surface through a balloon mold. In one or more embodiments, balloon molds can be etched or micro machined to create the micropattern.

Although the micropillars 112 in FIG. 43 are depicted as rectangular prisms, the present disclosure includes any suitable micropillar shape (e.g., shape of micropillars as shown in FIG. 44, cylindrical, elliptical prism, etc.) as described herein. Although micropillars 112 of FIG. 42 through 44 are arranged in longitudinal rows and circumferential columns, a micropatterned polymer coating 110 may include micropillars arranged in any of the wide variety of patterns described herein. The micropatterned polymer coating 110 may include any of the polymer coating materials described herein and may be manufactured by any of the manufacturing techniques described herein.

As shown, for example, in FIG. 45, in one or more embodiments, a micropattern polymer coating may be applied to a medical device (e.g., a variceal ligation band, etc.) to improve tissue retention. For example, ligation bands (e.g., variceal ligation bands) may be used to ligate tissue. In one or more embodiments, an esophageal variceal and EMR (endoscopic mucosal resection) bander may include a micropatterned polymer coating to improve tissue retention.

In one or more embodiments, a method of ligating tissue (e.g., esophageal varices, etc.) may include using a bander to generate a suction on tissue to be ligated. A band may be released over the tissue to be ligated (e.g., to form a pseudo polyp, to capture esophageal varices, etc.). In one or more embodiments, the suction may be released.

In an endoscopic mucosal resection, the tissue (e.g., a lesion) captured can be removed.

For variceal banding, the band would stay in place. In many cases, enlarged or swollen veins on the lining of the esophagus are prone to bleeding and can be life-threatening, and can be fatal in up to 50% of patients. Thus, improved ligation bands that do not prematurely slip off of the target tissue are desired.

In one or more embodiments, the surface tension of a ligation band may be varied in order to enhance the ligation bands retention to tissue.

As shown in FIG. 45, tissue 122 may be ligated by a band 120 of a ligator device. The cross sectional shape of the band 120 may be round (e.g., oval) as shown in FIG. 45 or may have, for example, a square cross sectional shape that assists in the retention of the band to the tissue 122. In one or more embodiments, the band 122 may include an outer surface having a surface area, wherein the outer surface 124 has a micropatterned polymer coating 126 thereon. In one or more embodiments, the micropattern may include micropillars that collectively enhance retention with the ligated tissue, which can reduce or eliminate the likelihood of the band slipping and/or rolling off of the captured tissue.

In one or more embodiments, the band surface 124 has a non-uniform surface tension. In one or more embodiments, a micropatterned polymer coating 124a may be disposed on an inner surface 128 of a band 122 (at, for example, the inside diameter of the band) as shown in FIG. 46, wherein the micropatterned polymer coating 124a is designed to enhance tissue retention. In one or more embodiments, a micropatterned polymer coating 124b may be disposed on an outer surface 130 of a band 122 (at, for example, the outside diameter of the band) as shown in FIG. 46, wherein the micropatterned polymer coating 124b is designed for friction reduction, which can facilitate deployment. Although the band shown in FIG. 46 has a four-sided (e.g., square, rectangle) cross-sectional area, the micropatterned polymer coatings 124a, 124b may be disposed on an inside surface and an outside surface, respectively, of a band 122 having any cross-sectional shape (e.g., a round shape as in FIG. 45) having any number of sides.

In at least one embodiment, a micropatterned polymer coating 126 may increase the surface tension of one or more portions of the band surface 124.

In at least one embodiment, a ligation band may include a micro-pattern on, for example, a square-, round-, or hexagonal-shaped cross sectional portion of the band in a pattern such as a spiral around a torus. In one or more embodiments, such a patterns can provide a one-way directional resistance to tissue, such that the pattern can be placed easily on tissue in one direction and can resist (e.g., through surface tension) inadvertent displacement in the other direction. In one or more embodiments, a micropattern can be selected to set an angle (e.g., of a spiral around a torus) to prevent rolling in a specific direction.

As shown, for example, in FIG. 47, in one or more embodiments of the present disclosure a medical device (e.g., a resection snare) may have a micropatterned polymer coating thereon.

Snares (e.g., hot cautery snares, etc.) have been used for various tissue resection procedures, including, but not limited to, endoscopic mucosal resection (EMR) and endoscopic submucosal dissection (ESD). Procedures and devices for use in EMR and ESD have been reported by Kantsevoy et al., “Endoscopic mucosal resection and endoscopic submucosal dissection,” Gastrointestinal Endoscopy, July 2008, 68(1): 11-18.

Some snares used for various tissue resection procedures have a tendency to slip off of tissue that is intended to be ensnared. In a typical endoscopic mucosal resection (EMR), a submucosal layer is injected with saline to lift the mucosal layer, thereby forming a bleb. A bleb can facilitate access to and visualization of a lesion, and creates a protective barrier over the muscularis. However, the formation of the bleb can sometimes make snaring more difficult because of an increase in tension in the mucosa surrounding the lesion. A bleb having a low profile may be more difficult to snare because of the propensity for the snare to slide up an edge of the bleb and slip off The geometry and volume of the bleb varies based on technique, solution properties, injection speed, and lesion morphology. A snare operator (e.g., a physician) may apply a downward force with an endoscope in an attempt to improve snare traction, but the lack of stiffness in the snare limits this force because the distal end of the snare loop tends to deflect away from the tissue plane. Improved snares for resecting tissues having a low profile are desired.

In one or more embodiments, an apparatus for tissue resection can include a snare loop having a micropatterned polymer coating thereon. In one or more embodiments, the micropatterned coating may be disposed on the portions of the snare intended to contact tissue during a resection. For example, the micropatterned polymer coating may be disposed on an inner perimeter surface of the snare (e.g., the surface that faces inside a snare loop). In one or more embodiments the micropatterned polymer coating can be disposed on and can extend around a longitudinal portion of the snare.

In one or more embodiments, a snare may include a micropatterned polymer coating to provide a surface that can facilitate tissue resection by providing adhesion between the snare (e.g., the coated snare wire) and tissue.

In at least one embodiment, a micropatterned polymer coating may include a surface with appropriately-spaced micro-features (e.g., micropillars) that can facilitate a conformal interface with wet, biological substrates (e.g., tissue, blebs, etc.).

In one or more embodiments, as depicted in FIG. 47, an apparatus 140 may include an elongate snare 142 (e.g., a hot cautery snare, a resection snare, etc.) having a micropatterned polymer coating 146 on a surface along one or more portions of the snare 142 or along the entirety of the snare (e.g., the entirety of a snare loop). For example, a micropatterned polymer coating may provide micropatterned polymer coating 146 applied to one or more distal shoulders 148 of the snare 142, where snares have been prone to slipping. In one or more embodiments, a micropatterned polymer coating can be disposed along a snare at any location where tissue adhesion is desired. In one or more embodiments, a micropatterned polymer coating may be disposed at a distal portion of the loop (e.g., the portion of the loop farthest from the snare operator).

In FIG. 48, an apparatus 140 includes an elongate snare 142 having a micropatterned coating 146. As the snare is retracted within a lumen 144, the micropatterned polymer coating 146 can grip tissue 150 (e.g., a bleb) without slipping along the tissue surface, which is shown having a low profile.

In FIG. 49, an apparatus 140 includes an elongate snare 142 having a micropatterned coating 146 that extends completely circumferentially around a distal segment of elongate snare 142. In one or more embodiments, a snare 142 may include a plurality of micropatterned polymer coatings disposed at one or more portions of the snare that may engage tissue.

In one or more embodiments, a snare loop can be constructed of several wound or braided wires, some or all of which could be individually covered by a micropatterned polymer coating having a micropatterned surface. In at least one embodiment, the micropatterned surface can adhere to tissue on contact and thereby reduces slippage of the snare when closing the snare.

A snare apparatus 140 may include a single elongate lumen 144, having a proximal handle end (e.g., for user actuation), and a retractable snare 142 (e.g., a snare loop). In one or more embodiments, the elongate lumen could be sized to fit down a working channel of an endoscope. In one or more embodiments, the snare loop could be of any configuration including but not limited to an oval, round, hex, or duckbill configuration. In one or more embodiments, a snare loop may include several elements (e.g., wires, etc.). In the present disclosure, a micropatterned polymer coating may be applied to any of the one or more elements of the snare that comes in contact with tissue.

In at least one embodiment, a micropattern (e.g., a micropatterned polymer coating) may be applied to one or more snare elements of an array of snare elements. In at least one embodiment, the micropatterned polymer coating can engage tissue and can reduce or prevent slippage during actuation and/or burning.

In one or more embodiments, an extrusion die is provided for manufacturing a wire (e.g., a filament, etc.) having a micropattern thereon. In one or more embodiments, the wire may be formed from a metal or alloy. In at least one embodiment, the wire is formed from steel. In one or more embodiments, the wire is formed from a polymer or composite. In one or more embodiments the extrusion die is structured and arranged to provide a micropattern to the wire. In at least one embodiment, the microstructure is formed integrally with the wire. In at least one embodiment, the microstructure is formed as a micropatterned polymer coating applied to the exterior surface of a wire.

As shown, for example, in FIG. 51, in one or more embodiments of the present disclosure, a medical device (e.g., a retrieval balloon) may have a micropatterned polymer coating thereon.

Catheter-mounted balloons have been used to retrieve stones (e.g., gall stones, kidney stones, etc.) and/or other foreign objects from various body lumens (e.g., the bile duct, urinary tract, etc.) by inflating a balloon at a location distal of a stone in order to obstruct the body lumen, followed by dragging (e.g., pulling) the inflated balloon in a proximal direction so that the balloon pushes the stone in a proximal direction ultimately to the end of the lumen (e.g., the lumen opening). The stones, however, have been reported to slip around the balloon as the balloon is pulled in a proximal direction. This is shown in FIG. 50, wherein balloon 160, which was inflated in a lumen 166 at a position distal of a stone 162, is pulled proximally by an operator pulling catheter 164. As shown, the wall 168 of lumen 166 may flex sufficiently to allow passage of stone 162 around balloon 160 (or, from another perspective, allow passage of balloon 160 passed stone 162) as the balloon 160 is pulled proximally.

In one or more embodiments, a retrieval balloon may have a micropatterned polymer coating thereon, wherein the micropatterned polymer coating can adhere to stones (e.g., gallstones, kidney stones, etc.) or other foreign objects.

For example, as depicted in FIG. 51, a retrieval device including an inflation catheter 164 (e.g., with guide wire rail) and a balloon 160 with a micropattern surface 170 (e.g., a micropatterned polymer coating) on the inflation catheter, the balloon, or both of the inflation catheter and the balloon. In one or more embodiments, the micropattern can be disposed on a proximal portion of the balloon or a portion of the catheter proximal of the balloon and may be selected to enhance adherence to stones. In one or more embodiments, the micropatterned polymer coating may extend proximally from the balloon to cover a portion of the catheter in addition to the balloon. In one or more embodiments, a plurality of noncontiguous micropatterned polymer coatings are present on the balloon, the catheter, or both.

In one or more embodiments, the adhesive micropatterned polymer coating 170 may inhibit or prevent the stone 162 from migrating laterally (e.g., across the lumen 166) as it is dragged through the body lumen 166. In one or more embodiments, the lateral-facing surfaces (e.g., the surfaces contacting the lumen when inflated) and distal-facing surfaces of the balloon 160 can optionally include an adhesive micropatterned polymer coating 170 and may optionally include a different micropatterned polymer coating having a micropattern surface designed and selected to facilitate sliding through the body lumen 166.

This micropatterning could apply to other devices that use balloons for retrieval, such as combination retrieval balloons and tomes.

In one or more embodiments, the adhesiveness of the micropatterned polymer coating may be modified based on, for example, the level of inflation (e.g., pressure) of the balloon. Although not wishing to be bound by theory, it is believed that the adhesive properties of the micropattern vary based, at least in part, on the spacing of the microfeatures (e.g., micropillars, etc.) and could therefore be designed to vary based on the level of inflation of the balloon. That is, inflation of the balloon can, in some embodiments, stretch the balloon wall (e.g., axially and/or circumferentially) such that the area density of a given number of microfeatures (e.g., micropillars) would be reduced. In one or more embodiments, a balloon having a micropatterned polymer coating thereon may be inflated to stretch the balloon wall (or may be deflated to reduce stretching of the balloon wall) such that a micropatterned surface on the balloon wall exhibits a change in hydrophobicity (or hydrophilicity). In one or more embodiments, varying pressure in a balloon having a micropatterned polymer coating thereon can cause a transition from hydrophobic to hydrophilic states as the surface of the balloon expands or retracts.

As shown, for example, in FIG. 53, in one or more embodiments of the present disclosure, a medical device (e.g., an apparatus for stone retrieval) may have a micropatterned polymer coating thereon.

In the medical field, a basket has been used endoscopically to grasp, manipulate, and/or remove calculi and other foreign objects from one or more body lumens (e.g., tracts in the body) including, but not limited to, the urinary tract. It is common to use a basket for retrieval procedures. Operators of retrieval baskets (e.g., physicians) have experienced some difficulty in capturing and retaining the calculi stone. In a typical stone retrieval procedure, an operator uses a basket, which may contain four or more strains of wire that can be opened, closed, rotated, and/or longitudinally placed to capture a calculi stone. As shown in FIG. 52, basket system 180 includes a handle 182, which allows an operator to manipulate a basket 184 through an endoscopic device 186, which may be less than, equal to, or greater than about 120 centimeters long. In FIG. 52, basket 184 includes four wires 188, which may be connected at apex 190. The wires 188 form a basket 184 having windows 192 (e.g., between the wires) to allow a stone (not shown) to enter the basket 184. The basket 184 is then reduced (e.g., the wires are brought closer together, the windows are made smaller, etc.) to capture the stone within the basket 184. It is not uncommon for a stone to exit a window 192 during the closure procedure. Thus, improved basket devices for retrieving stones and/or other foreign objects from a body lumen are desired.

Referring to FIG. 53, a retrieval basket 184 has one or more micropattern surfaces (e.g., micropatterned polymer coatings 194) that can facilitate stone capture by providing adhesion between the basket wire 188 and a calculi stone. A micropatterned polymer coating 194 having a micropatterned surface with appropriately-spaced microfeatures (e.g., micropillars) can create a conformal interface with wet, calculi substrates in one or more embodiments.

In one or more embodiments, a basket system includes a retrieval basket 184 having a micropattern surface (e.g., a micropatterned polymer coating 194) along one or more portions of the basket surfaces or on the entirety of the basket 184 surfaces. For example, the micropatterned polymer coating 194 surface could be applied to the inner surfaces of one or more wires 188 to adhere to a stone and/or prevent the stone from exiting through a window 192.

In one or more embodiments, the basket 184 could be formed from one or more metals, alloys, oxides, polymers, composites, or mixtures thereof, such as, but not limited to, Nitinol, stainless steel, cobalt, chrome, and others. In at least one embodiment, the micropatterned polymer coating could be applied by any of a wide variety of manufacturing techniques described herein including extrusion, compression dies, or over molding configurations.

As shown, for example, in FIG. 54, in one or more embodiments of the present disclosure, a medical device (e.g., a bronchial thermoplasty catheter) may have a micropatterned polymer coating thereon.

Bronchial thermoplasty has been used as a treatment for severe asthma and involves delivery of a controlled, therapeutic radiofrequency energy to an airway wall, thereby heating the tissue and reducing the amount of smooth muscle present in the airway wall.

An exemplary bronchial thermoplasty catheter device 200 is depicted in FIG. 54. During a bronchial thermoplasty procedure, mucus secretions are known to stick to all areas of a catheter's electrode array, which inhibits endoscopic vision and/or creates a need for a user to pause the procedure, remove the catheter, and clean the array before continuing with the procedure. Mucus is also known to build up in the airway, which creates a need for a user to pause the procedure, remove the catheter, and perform suction through the bronchoscope. Thus, improved bronchial thermoplasty catheter devices are desired that could, for example, prevent or reduce mucus build up on the electrode array and facilitate the ability to perform suction without removing the catheter from the bronchoscope.

During a bronchial thermoplasty procedure, getting sufficient contact between all electrodes and the airway wall has been known to be difficult due to, for example, the bronchoscope position in the airway, or branching or curved airways, which can cause incomplete activations or inconsistent treatment. Maintaining contact during a breathing cycle has also been a difficulty because airway movement occurs, which can cause incomplete activations or inconsistent treatment. Thus improved bronchial thermoplasty catheter devices are desired that could, for example, improve contact between the active electrode region and the airway wall.

In one or more embodiments, a bronchial thermoplasty catheter device includes a micropattern (e.g., a micropatterned polymer coating), which may offer one or more benefits.

For example, as shown in FIG. 54, a micropatterned polymer coating having a hydrophobic (e.g., slippery) micropattern may be applied to one or more of the following: distal and/or proximal electrode insulation on one or more of the electrodes A, distal tip B, distal and/or proximal alignment extrusion C, shaft D, and electrodes E (e.g., active electrode region). In one or more embodiments, a hydrophobic micropattern can prevent buildup of mucus on the array 202 (of electrodes E), to improve endoscopic view, and/or to reduce the need to remove the catheter to remove mucus from the device, and/or to reduce or prevent mucus bridges that can create an unintended electrical path. In one or more embodiments, a hydrophobic micropattern can allow suction of mucus through a bronchoscope while the catheter is in place.

In one or more embodiments, an electrically conductive conformal (e.g., sticky) micropattern on the active electrode regions of one or more electrodes can improve electrode contact with an airway wall and can maintain contact when movement of the airway wall occurs due to, for example, the breathing cycle.

In one or more embodiments, the electrode array 202 could be formed from any conductive material and may include, but is not limited to, Nitinol, stainless steel, cobalt, chromium (e.g., chrome), or a shape memory polymer. In at least one embodiment, the micropatterned polymer coating could be applied by any of a wide variety of manufacturing techniques described herein including extrusion, compression dies, electro deposition, photoetching, or over molding configurations.

As shown, for example, in FIG. 55, in one or more embodiments of the present disclosure, a medical device (e.g., an endoscope system) may have a micropatterned polymer coating thereon.

Referring to FIG. 55, an endoscope device 380 can include vision capabilities and a working channel 382 through which catheters may be advanced and withdrawn, a visual channel 384, and an injection channel 386. Some endoscope devices may be controlled using pull wires 388 and knob controls (not shown) at or near the proximal end of the device. Some endoscopic devices include an occlusion balloon 390, which may include a radiopaque marker 392 disposed thereunder.

In or more embodiments, an endoscope device can include an occlusion balloon having a micropatterned surface (e.g., a micropatterned polymer coating) thereon. A micropatterned surface on the balloon 390 can improve anchoring of an endoscope device 380 inside the distal end on the papilla, where there is a tortuous scope angle and a short landing area within the duct. In one or more embodiments, the improved ability to anchor in this anatomy may facilitate a stone or stricture procedure.

In one or more embodiments, the balloon has a microstructure designed to adhere to a bile duct wall. In some embodiments, the catheter portion near the balloon may also have a micropattern surface. In some embodiments, the adhesion to tissue may prevent migration laterally as a stone or stricture procedure is performed within the lumen.

In one or more embodiments, the distal portion of the balloon need not have a micropattern thereon or may include a micropattern surface designed to facilitate sliding through the anatomical lumen.

In one or more embodiments, an adhesive micropattern may be applied to balloons used for anchoring, such as coronary and peripheral interventions associated with placing stents.

As shown, for example, in FIG. 56, in one or more embodiments of the present disclosure, a medical device (e.g., a bronchiole occluder coil) may have a micropatterned polymer coating thereon.

Embolization coils have been used to treat aneurysms. Embolization coils have combined a microcatheter and a wire, wherein the wire is deposited via the microcatheter within a vascular cavity to form an occlusion.

Emphysema is categorized by having enlarged alveoli having weakened air sacs and excess mucus. Additionally, having fewer capillaries around these structures have made it difficult to expel oxygen-depleted air from these areas of the lungs.

In one or more embodiments of the present disclosure, a method for treating emphysema is provided. The method includes occluding a diseased bronchiole. In one or more embodiments, the method includes disposing a wire (e.g., a releasable occluder coil) within a bronchiole and/or alveoli (e.g., in a lung), wherein the wire has a micropatterned polymer coating thereon. In one or more embodiments, the occluder coil could be of a similar size as that used to treat 3 millimeter aneurysm within the bronchiole.

Referring to FIG. 56, a microcatheter 220 having a lumen terminating at a distal opening extends through an airway and into a bronchiole (see inset). In one or more embodiments, a wire 222 is passed through the microcatheter lumen and released from the distal opening at a location within a bronchiole 224 or alveoli 226. In one or more embodiments, the wire 222 is an occluder coil structured and arranged to occlude a bronchiole 224 and/or an alveoli 226.

In at least one embodiment, a wire 222 to be disposed could be an occluder coil that releases more distally into the alveoli 226, having a release diameter of, for example, less than about 1 millimeter. It should be recognized that the diameter of the wire 222 to be disposed can be selected based on the target site for releasing the occluder coil. In one or more embodiments, a wire may include a plurality of portions having different diameters, wherein a relatively smaller diameter portion can be deposited at a site more distal, whereas the larger diameter portion may extend into a site more proximal.

For example, one or more embodiments of a method include use of an occluder coil having a release diameter of 1 millimeter to occlude an alveoli with a fine wire. In one or more embodiments, an occluder coil can include a gradual transition to 4-millimeter release diameters to be disposed in fine bronchiole. In one or more embodiments, an occluder coil for occluding a large bronchiole structures can include a 12-millimeter release diameter or an even greater wire diameter.

In one or more embodiments, an occluder coil could contain fillers such as a plurality of polyester filamentary hairs and/or other fragments.

In one or more embodiments, an occluder coil or a filler fragment could include a micropattern (e.g., a micropatterned polymer coating) thereon to help engage the occluder coil to the bronchiole structures.

In one or more embodiments, an embolization coil for use in treating an aneurism (e.g., for placement within a vascular cavity) may include a micropatterned polymer coating therein to help engage the vascular cavity tissue.

In one or more embodiments, an adhesive micropatterned surface (e.g., micropatterned polymer coating) can adhere to tissue, thereby preventing the occluder coil (e.g., a micro coil) from migrating out of or being expelled out by a bronchiole lumen. In one or more embodiments, the pillars (e.g., micropillars) of the micropattern may be arranged having an angular orientation with respect to the micropattern base which, in some embodiments, can reduce the force required for initial pushing and release (i.e., delivery) from a microcatheter due to the micropattern having only one-way directional resistance.

As shown, for example, in FIG. 57, in one or more embodiments of the present disclosure, a medical device (e.g., a biopsy needle) may have a micropatterned polymer coating thereon.

Endoscopic ultrasound fine needle aspiration (EUS FNA) is a well-established procedure (e.g., in the fields of cytology, histology, etc.), but one in which improvements in sensitivity and specificity are constantly sought. Various gauge sizes are utilized with 25, 22, and 19 gauge being the most common.

In one or more embodiments, a micropatterned polymer coating may be applied to a biopsy needle in order to, for example, improve sampling.

For example, with reference to FIG. 57, a portion of an exemplary biopsy needle 230 is depicting, wherein the needle 230 has an inner surface 240 defining a lumen 232 that terminates at a lumen opening that includes a perimeter including a needle tip 234 and a needle heel 236. In one or more embodiments, the needle inner surface 240 has one or more micropatterned polymer coatings 242 thereon.

In one or more embodiments, the micropatterned polymer coating 242 can be applied to the inner surface 240 of a biopsy needle 230 for the purpose of abrading and/or shaving off additional cellular material during a sample acquisition.

In one or more embodiments, the micropattern architecture would include micropillars biased toward the needle point 234 such that the longitudinal (e.g., back and forth) motion typically employed during endoscopic ultrasound fine needle aspiration would lead to increased abrasion and would free additional cellular tissue that could then be aspirated into the needle.

In one or more embodiments, a micropatterned polymer coating could be applied to additional needle grinds. For example, on a 3-point Franseen grind (commercially available from Hart Enterprises Inc. (Sparta, Mich.)), a micropatterned polymer coating could be applied on the inner surface of each of three points causing cells to be released both on the points as well as the three heels.

In one or more embodiments, the stylet component of a Franseen needle would include a lubricious micropattern (e.g., a micropatterned polymer coating) to inhibit or avoid picking up cells and to reduce or prevent the micropatterned polymer coating on the inner surface from picking up tissue material until the stylet was removed.

The heel 236 of the needle 230 generally provides an edge for sampling. A micropatterned polymer coating may be applied to the heel of the needle to provide improved sampling.

In one or more embodiments, the micropattern of the micropatterned polymer coating can be biased away from the needle point, which would promote sample retention as well as potentially promoting core biopsies. In one or more embodiments, a needle 230 may include one or more windows (e.g., ground windows) separated by a distance from the needle grind, each of which would provide an additional heel-type grind as well allowing for the micropatterned polymer coating to be applied deeper into the needle. In at least one embodiment, a micropatterned polymer coating can be applied to the outside surface 238 of the needle 230 such that cellular samples could be retained on the outer diameter as well as the inner diameter.

In one or more embodiments the microfeatures (e.g., micropillars) may be sized (e.g., dimensioned) and arranged within a micropattern having dimensions that are suited to retain one or more specific tissue types. For example, a micropattern may be selected to specifically retain blood cells. For example, in some embodiments, it may be preferred that the blood is retained on the needle rather than being expelled onto a slide or sample jar. This would be particularly relevant on, e.g., either larger gauge needles that would be less likely to become clogged or if applied to the outer diameter. In one or more embodiments, a micropattern could be sized/dimensioned such that blood cells would not be retained, but smaller cells would be retained.

In one or more embodiments, one or more additional micropatterned polymer coatings can be applied to a needle surface (e.g., an inner surface, an outer surface, a needle tip, a needle tip perimeter, etc.) for purposes of adhesion and/or anti-adhesion according to a particular application.

As shown, for example, in FIG. 59, in one or more embodiments of the present disclosure, a medical device (e.g., a retracting strap and grasper) may have a micropatterned polymer coating thereon.

During a surgical procedure, a surgical device should be able freely move, articulate, and approach a clear area of interest as is needed to perform the required procedures and complete the tasks as quickly and safely as possible. However, for some surgical procedures, body tissues may obstruct the free movement, articulation, and movement of surgical devices. Improved medical devices are desired that may obstruct a surgical procedure.

For example, in one or more embodiments, a retractor strap can include one or more micropatterned gripping surfaces (e.g., a micropatterned polymer coating) to grip tissues one or more body tissues. As depicted in FIG. 58, a retractor strap system 250 includes a strap 252 having a micropatterned polymer coating 260 (FIG. 59) disposed at or near each of a first end 254 and a second end 256 of the strap 252. In at least one embodiment, the strap may be positioned to secure one or more tissues against another one or more tissues to which each of the micropatterned polymer coatings are adhered. A retractor strap system of the present disclosure may be used during a surgical, therapeutic, or other type of procedure, such that access to a target area may be made free of any obstructions for a clear view and easy access for a safe, quick, and effective procedure.

In one or more embodiments a retractor strap system may be used to creating work space or volume or to retract tissues in order to work in a confined space of a body lumen, a body cavity (e.g., the abdominal cavity) or other limited space area.

In one or more embodiments, the strap 252 can be structured and arranged in order to be delivered (e.g., through a Working Channel (WC) of an endoscopic device or by other means) to a target site, applied to a target tissue (e.g., with a grasper or by other means). In one or more embodiments, straps 252 could have specific lengths and/or may be adjustable (e.g., with a size-adjusting, ratcheting pull tab 258 (FIG. 58) to tighten or loosen the restraining retracting strap). In at least one embodiment, a retractor strap system 250 could be removable, movable, re-usable, and/or implantable (e.g., short-term or long-term) and may include one or more absorbable materials.

In some embodiments, a retractable strap system 250 may be structured and arranged to be passed through a working channel (e.g., within an over sleeve or other means, wherein the over sleeve is removed when the retractable strap system is ready to use). In at least one embodiment, for example, a retractable strap system 250 can be rolled up and deployed through working channel. After the retractable stent system exits the working channel, the strap can be unrolled and positioned. In one or more embodiments, in addition to one or more micropatterned polymer coatings, a strap may include, e.g., Nitinol like support structure springs and/or beams to hold it in place or the strap could have a portion that is sutured, tacked or clipped in place (e.g., sutures, tacks, and clips may be temporary, releasable, absorbable, permanent, etc.).

Although the retractor strap systems 250 of FIG. 58 and FIG. 59 include two micropatterned polymer coatings, the present disclosure contemplates retractor strap systems having multiple (e.g., 3, 4, 5, more than 5, etc.) legs, each of which has a micropatterned polymer coating thereon, which may be useful to retract a large organ. In one or more embodiments, a plurality of retractable strap systems may be used to collectively retract tissue (e.g., a large organ).

In some procedures, delicate or thin tissue may need to be grasped. There is an on-going need for improved devices for grasping tissue without causing undue trauma.

In one or more embodiments, as shown in FIG. 60, an end effector grasper or clip 270 can include a micropatterned polymer coating 272 on, e.g., one or more jaws. In one or more embodiments, a micropatterned polymer coating can be less traumatic on delicate thin tissue. In one or more embodiments, an end effector having a micropatterned polymer coating thereon may be included on the trap ends (e.g., of a retractor strap system) to grasp tissues.

In one or more embodiments, strap 252 can have any suitable dimensions or shape (e.g., circle, oval, square, rectangle, irregular, etc.).

In one or more embodiments, a medical device having one micropatterned polymer coating may be useful in one or more medical applications (e.g., endoscopic mucosal resection, endoscopic submucosal dissection, etc.) for gripping tissue. For example, in one or more embodiments, a medical device can include a strap (or any structure that can support a tension) having a micropatterned polymer coating thereon. In at least one embodiment, the micropatterned polymer coating may be adhered to tissue having, for example, a low profile, wherein the tissue is to be ligated. After the micropatterned polymer coating is adhered to the tissue, the strap may be pulled or otherwise manipulated to lift the tissue (e.g., a lesion) in order to raise the profile of the tissue to be ligated. After the tissue is raised, the tissue may be ligated with any appropriate cutting tool (e.g., a needle, a knife, snare, etc.).

A description of some exemplary embodiments of the present disclosure is contained in one or more of the following numbered statements:

Statement 1. A medical device comprising:

a catheter;

a balloon mounted on the catheter, the balloon extending longitudinally from a distal end to a proximal end and having an outer surface, the balloon radially expandable from a first state wherein the balloon has a first diameter to an inflated state wherein the balloon has a second diameter greater than the first diameter;

wherein the balloon, in the inflated state, defines a laterally-facing body portion that extends along the portion of the balloon having the largest diameter, the lateral-facing body portion comprising a lateral-facing surface, wherein the outer surface of the balloon comprises:
the lateral-facing surface,
a distal-facing surface extending from the distal end of the lateral-facing surface to the distal end of the balloon and having a diameter less than the lateral-facing surface, and
a proximal-facing surface extending from the proximal end of the lateral-facing surface to the proximal end of the balloon and having a diameter less than the lateral-facing surface,

wherein at least a portion of the outer surface of the balloon comprises a micropattern.

Statement 2. The medical device of statement 1, wherein the micropattern comprises an adhesive micropattern.

Statement 3. The medical device of statement 1 or statement 2, wherein at least a portion of the catheter comprises a micropattern.

Statement 4. The medical device of any one of statements 1-3, wherein a portion of the catheter that is proximal of the balloon comprises a micropattern.

Statement 5. The medical device of any one of statements 1-4, wherein the micropattern of the catheter and the micropattern of the balloon are contiguous.

Statement 6. The medical device of any one of statements 1-5, wherein the proximal-facing surface comprises a micropattern.

Statement 7. The medical device of statement 6, wherein the distal-facing surface does not include a micropattern.

Statement 8. The medical device of statement 6 or statement 7, wherein the lateral-facing surface does not include a micropattern.

Statement 9. The medical device of any one of statements 6-8, wherein at least one of the proximal-facing surface and the lateral-facing surface comprises a micropattern that is different from the micropattern of the proximal facing surface.

Statement 10. The medical device of any one of statements 1-9, wherein the catheter and balloon are structured and arranged to extend within a body lumen.

Statement 11. The medical device of any one of statements 1-10, wherein the balloon is structured and arranged to slide through the body lumen while at least a portion of the balloon has the same diameter as the body lumen.

Statement 12. The medical device of any one of statements 1-11, wherein the at least a portion of the outer surface comprises the lateral-facing body portion, and wherein the micropattern is designed and selected to increase adhesion with a body lumen in which the balloon is inflated.

Statement 13. Method of removing a foreign object from a lumen comprising:

providing a medical device of any one of statements 1-12;

disposing the balloon in a lumen, having a foreign object disposed therein, at a location distal of the foreign object;

inflating the balloon to obstruct the lumen;

moving the balloon proximally through the lumen to remove the foreign object from the lumen.

Statement 14. The method of statement 13, wherein moving the balloon proximally through the lumen comprises contacting the foreign object with the micropattern.

Statement 15. The method of statement 13 or statement 14, wherein moving the balloon proximally through the lumen comprises adhering the foreign object to the balloon or the catheter via the micropattern.

Statement 16. The method of any one of statements 13-15, wherein the foreign object is a kidney stone or a gall stone.

Statement 17. The method of any one of statements 13-16, wherein the lumen is a bile duct or urinary tract.

Statement 18. The method of any one of statements 13-17, wherein the micropattern comprises a plurality of noncontiguous micropatterned polymer coatings on the balloon, the catheter, or both.

Statement 19. The method of any one of statements 13-8, wherein the micropattern comprises an adhesive micropattern that, upon contact with the foreign object, inhibits or prevents the foreign object from migrating laterally as it is moved proximally through the lumen.

Statement 20. The method of any one of statements 13-19, wherein the lateral-facing body portion comprises a micropattern designed and selected to facilitate sliding through the lumen.

The above disclosure is intended to be illustrative and not exhaustive. This description will suggest many variations and alternatives to one of ordinary skill in this art. All these alternatives and variations are intended to be included within the scope of the claims where the term “comprising” means “including, but not limited to.” Those familiar with the art may recognize other equivalents to the specific embodiments described herein which equivalents are also intended to be encompassed by the claims.

Further, the particular features presented in the dependent claims can be combined with each other in other manners within the scope of the present disclosure such that the present disclosure should be recognized as also specifically directed to other embodiments having any other possible combination of the features of the dependent claims.

This completes the description of the preferred and alternate embodiments of the present disclosure. Those skilled in the art may recognize other equivalents to the specific embodiment described herein which equivalents are intended to be encompassed by the claims attached hereto.

What is claimed is:

1. A medical device comprising:
a catheter;
a balloon mounted on the catheter, the balloon extending longitudinally from a distal end to a proximal end and having an outer surface, the balloon radially expandable from a first state wherein the balloon has a first diameter to an inflated state wherein the balloon has a second diameter greater than the first diameter;
wherein the balloon, in the inflated state, defines a laterally-facing body portion that extends along the portion of the balloon having the largest diameter, the lateral-facing body portion comprising a lateral-facing surface, wherein the outer surface of the balloon comprises:
the lateral-facing surface,
a distal-facing surface extending from the distal end of the lateral-facing surface to the distal end of the balloon and having a diameter less than the lateral-facing surface, and
a proximal-facing surface extending from the proximal end of the lateral-facing surface to the proximal end of the balloon and having a diameter less than the lateral-facing surface,
wherein at least a portion of the outer surface of the balloon comprises a micropattern.

2. The medical device of claim 1, wherein the micropattern comprises an adhesive micropattern.

3. The medical device of claim 1, wherein at least a portion of the catheter comprises a micropattern.

4. The medical device of claim 1, wherein a portion of the catheter that is proximal of the balloon comprises a micropattern.

5. The medical device of claim 1, wherein the micropattern of the catheter and the micropattern of the balloon are contiguous.

6. The medical device of claim 1, wherein the proximal-facing surface comprises a micropattern.

7. The medical device of claim 6, wherein the distal-facing surface does not include a micropattern.

8. The medical device of claim 6, wherein the lateral-facing surface does not include a micropattern.

9. The medical device of claim 6, wherein at least one of the proximal-facing surface and the lateral-facing surface comprises a micropattern that is different from the micropattern of the proximal facing surface.

10. The medical device of claim 1, wherein the catheter and balloon are structured and arranged to extend within a body lumen.

11. The medical device of claim 10, wherein the balloon is structured and arranged to slide through the body lumen while at least a portion of the balloon has the same diameter as the body lumen.

12. The medical device of claim 11, wherein the at least a portion of the outer surface comprises the lateral-facing body portion, and wherein the micropattern is designed and selected to increase adhesion with a body lumen in which the balloon is inflated.

13. Method of removing a foreign object from a lumen comprising:
providing a medical device of claim 1;
disposing the balloon in a lumen, having a foreign object disposed therein, at a location distal of the foreign object;
inflating the balloon to obstruct the lumen;
moving the balloon proximally through the lumen to remove the foreign object from the lumen.

14. The method of claim 13, wherein moving the balloon proximally through the lumen comprises contacting the foreign object with the micropattern.

15. The method of claim 13, wherein moving the balloon proximally through the lumen comprises adhering the foreign object to the balloon or the catheter via the micropattern.

16. The method of claim 13, wherein the foreign object is a kidney stone or a gall stone.

17. The method of claim 13, wherein the lumen is a bile duct or urinary tract.

18. The method of claim 13, wherein the micropattern comprises a plurality of noncontiguous micropatterned polymer coatings on the balloon, the catheter, or both.

19. The method of claim 13, wherein the micropattern comprises an adhesive micropattern that, upon contact with the foreign object, inhibits or prevents the foreign object from migrating laterally as it is moved proximally through the lumen.

20. The method of claim 13, wherein the lateral-facing body portion comprises a micropattern designed and selected to facilitate sliding through the lumen.
